(19) United States
(12) Patent Application Publication
(10) Pub. No.: US 2011/0087206 A1
(43) Pub. Date: Apr. 14, 2011

(54) CRYOSURGICAL DEVICE FOR OPERATING CRYOPROBES, METHOD FOR OPERATING A CRYOPROBE

(76) Inventors: Franz Geiselhart, Reutlingen (DE); Stefan Gross, Tubingen (DE)

(21) Appl. No.: 12/997,532

(22) PCT Filed: May 13, 2009
(86) PCT No.: PCT/EP2009/003407

§ 371 Date: Dec. 10, 2010

(30) Foreign Application Priority Data
Jun. 12, 2008 (DE) 10 2008 028 046.1
Aug. 19, 2008 (DE) 10 2008 038 310.4

(51) Int. Cl. A61B 18/02; A61F 7/10

(52) U.S. Cl. 606/23; 607/104

(57) ABSTRACT

A cryosurgical apparatus capable of operating both a first cryoprobe with a pressure-resistant return run and a second cryoprobe with an unpressurized return run. The apparatus has a first mode for operating the first cryoprobe and a second mode for operating the second cryoprobe. The apparatus includes a controller, a fluid source for providing a fluid for cooling the cryoprobes by of the Joule-Thomson effect, at least two cryoprobe connections one of which is connected to the first and second cryoprobe in the first and second mode, respectively, and a pressure-setting device with at least one proportional valve. The pressure-setting device can regulate, in the first and second modes, the pressure ratio of the admission run to the return run of the cryoprobe and the pressure in the admission run of the cryoprobe, respectively. The controller controls the cooling power of the cryoprobes by way of setting the proportional valve.

### FIELD OF THE DISCLOSED EMBODIMENTS

The disclosed embodiments relate to a cryosurgical apparatus for operating a first cryoprobe with a pressure-resistant return run and a second cryoprobe with an unpressurized return run and also to a method of operating such a cryosurgical apparatus.

### BACKGROUND

Cryotherapy, including cryosurgery, has a large number of applications. Cryoprobes are used, for example, to destroy diseased tissue, to take tissue samples and/or to remove foreign bodies. In cryotherapy, and in particular in cryosurgery, cold is frequently applied by means of a probe in order to achieve a healing effect.

There are various methods that may be used to cool the corresponding instrument. The Joule-Thomson effect is frequently utilized for cooling. When cooling by the Joule-Thomson effect, a fluid, in particular a gas, is expanded near the site of application via a nozzle, and as a result of the expansion the gas experiences a change in temperature. The cooling power is based, inter alia, on the difference in pressure present at the site of the expansion. The expansion takes place in an expansion chamber. In order to ensure effective cooling of the instrument, it is necessary to remove the expanded gas from the expansion chamber without causing undesirable congestion. Gas is brought to the site of application via an admission run and removed from via a return run.

In practice, a distinction is drawn between two types of probes used for cooling: rigid defrostable probes and flexible probes. Rigid probes have a rigid, pressure-resistant admission run and return run. Advantageous regulation of the cooling power may be ensured at high and constant admission run pressure by the setting of the return run pressure. An advantage of rigid probes is that they may also be used for heating the site of application.

Flexible probes have a pressure-resistant admission run and a non-pressure-resistant return run. The expanded gas is sometimes drawn back inside the probe shank. Probes of this type are frequently much more flexible than a corresponding rigid probe. They may advantageously be used with flexible endoscopes or flexoscopes. However, the composition of the return lines or return guides of the fluid is such that these can only handle a low dynamic pressure. Therefore, the cooling power is regulated by flexible probes via the admission run pressure. After the expansion of the fluid in the expansion chamber, the fluid can flow away with low flow resistance (unpressurized return run).

It is desirable to be able to operate both types of probes on a single apparatus. A corresponding cryosurgical apparatus conventionally includes a fluid source, for example a gas cylinder containing a working gas (for example CO₂ or N₂O), a pressure-setting means for setting a suitable pressure on the admission run and/or return run and a controller which controls the pressure-setting device in such a way as to provide a constant and/or reproducible cooling power.

A corresponding cryosurgical apparatus which can operate both rigid and flexible probes is known from DE 10 2006 003 571 A1. However, in this device, the regulation of the cooling power of the connected probes by the apparatus is insufficient. Using this device, it is not possible to react to different supply pressures and temperature-induced changes.

### SUMMARY

Starting from the above described prior art, the object of the disclosed embodiments is to provide a cryosurgical apparatus for efficiently controlling cryoprobes that is embodied in a simple manner while at the same time complying with stringent safety standards. Furthermore, a method of operating such a cryosurgical apparatus is also discussed.

Disclosed embodiments include a cryosurgical apparatus for operating a first cryoprobe with a pressure-resistant return run and a second cryoprobe with an unpressurized or non-pressure-resistant return run, wherein the cryosurgical apparatus has a first mode, namely a back pressure regulating mode for operating the first cryoprobe, and a second mode, namely a front pressure regulating mode for operating the second cryoprobe. The apparatus includes a controller; a fluid source for providing a fluid, in particular a gas, which can be introduced, for cooling the cryoprobes, in particular by means of the Joule-Thomson effect, into the cryoprobes; at least two cryoprobe connections, there being connected to a cryoprobe connection of the cryoprobe connections in the first mode the first cryoprobe and in the second mode the second cryoprobe; and a pressure-setting device with at least one pressure regulating valve which is connected to the cryoprobes in such a way that the cryoprobes can be used to regulate, in both the first and second modes, the pressure ratio of the admission run to the return run of the cryoprobes and/or the pressure in the admission run, the controller controlling the cooling power of the cryoprobes by way of a setting of the pressure regulating valve.

The disclosed embodiments thus provide a fluid circuit which is as simple as possible and allows both flexible and rigid cryoprobes or cryoprobes with either pressure-resistant or non-pressure-resistant return runs to be operated. Furthermore, according to the disclosed embodiments, at least one cryoprobe connection is used for both types of probe. The complexity when allocating the individual cryoprobe plugs to the corresponding cryoprobe connections may thus be reduced.

According to the disclosed embodiments, a single pressure-setting device with a pressure regulating valve can be used to regulate both the fluid pressure in the return run of the first cryoprobe and that in the in the admission run of the second cryoprobe. Thus, the construction of the cryosurgical apparatus may be simplified. That affects not only the costs but also the safety of the apparatus. It should also be noted that the term “unpressurized” is to be interpreted herein to mean that an approximately atmospheric pressure is present. The pressure regulating valve may be a proportional valve or a needle valve.

In the case of the first cryoprobe, defrosting of the probe may be ensured if the pressure ratio of the admission run to the return run is set appropriately.

The pressure-setting device is connected in the first mode to the first cryoprobe such that the pressure in the admission run and in the return run of the first cryoprobe can be regulated by way of the setting of the pressure regulating valve. The aforementioned advantageous regulation of the cryoprobe power along the boiling-point/dew-point curve of the fluid (e.g., cooling by the Joule-Thomson effect) may in this way be ensured. Further advantages are a low flow speed, low flow resistance, a low loss of pressure on the admission and return run and better heat exchange effects on the probe head.

The pressure-setting device is connected in the second mode to the second cryoprobe such that, in the case of substantially—usually low—pressure in the return run of the second cryoprobe, the pressure in the admission run can be set. The cooling power of flexible probes may therefore also advantageously be set.

The pressure-setting device can include a 3/2 proportional valve (a valve with three connections and two main adjusting positions) which is connected by a first proportional valve connection or pressure regulating valve connection to the fluid source, by a second proportional valve connection or pressure regulating connection to the ventilation means and by a third proportional valve connection or pressure regulating valve connection to at least one of the cryoprobe connections. Preferably, this at least one cryoprobe connection is the connection used both by the first cryoprobe and by the second cryoprobe. As a result of the use of a 3/2 proportional valve, the pressure-setting device may be constructed in a very simple manner. The 3/2 proportional valve serves to set the pressure in the return run of the first cryoprobe and the volume of gas supplied into the admission run of the second cryoprobe.

The cryosurgical apparatus can include a switching device with at least one switching valve, which is connected to the pressure-setting device and at least one cryoprobe connection for changing between the first and the second mode.

Preferably, this cryoprobe connection is also the cryoprobe connection connected both to the first probe and to the second probe. The switching valve establishes a connection between the cryoprobe connection and the pressure-setting device in such a way that it is possible to set, in the first mode, the pressure in the return run and, in the second mode, the pressure in the admission run by way of the pressure-setting means, in particular by way of the pressure regulating valve therein.

The controller can activate the switching device. It is thus possible to ensure automatic changing between the control states, i.e. the first mode and the second mode. The doctor or the operating person does not have to take care to set the appropriate mode when connecting a first or second cryoprobe.

This is particularly advantageous if the plug-in connections to the probes are configured such that a corresponding allocation is carried out in accordance with the type of probe. It is possible to produce the switching device by way of a plurality of, in particular two, 2/2 switching valves (a valve with two connections and two adjusting positions). It is however more advantageous if the switching device comprises a 3/2 switching valve (three connections, two adjusting positions) which is connected by a first switching valve connection to the pressure-setting device, by a second switching valve connection to the ventilation means and by a third switching valve connection to a cryoprobe connection. The construction of the device may thus be further simplified.

The cryosurgical apparatus can include at least one pressure sensor for determining an input pressure and/or an output pressure of the pressure-setting means. Automatic regulating of the cooling power by way of the controller is thus possible.

Preferably, the fluid is made up of carbon dioxide and/or nitrous oxide (laughing gas) or a mixture of these gases. These gases have a high Joule-Thomson coefficient and are liquefiable at normal temperature. However, use may also be made of any other gas having a Joule-Thomson inversion temperature above the patient's body temperature.

The cryosurgical apparatus can include a detection means which ascertains whether a first cryoprobe or a second cryoprobe is connected to the cryoprobe connections. The detection means is connected to the controller and sends apparatus mark signals which allow the controller to ascertain whether a cryoprobe of the first type (first cryoprobe) or of the second type (second cryoprobe) is connected. This further increases the operator convenience of the apparatus. The controller can thus determine which cryoprobe connections are occupied and set the appropriate mode. The detection means may also serve as a securing means which detects the presence of a pneumatic contact between at least one cryoprobe connection and a cryoprobe. If the pneumatic connection is interrupted, then the securing means interrupts the supply of fluid. The issuing of fluid into the environment may be efficiently avoided in this way.

The cryosurgical apparatus can have a flow sensor for determining the cooling power of the connected cryoprobe. By determining the amount of fluid which flows through the cryoprobe, it is possible to draw conclusions about the cooling power applied in the cryoprobe. It is also possible to set or to determine the temperature by regulating and measuring the amount of fluid.

The disclosed embodiments also include a method for operating a cryoprobe with a cryosurgical apparatus, wherein the apparatus provides a fluid for cooling the cryoprobe. The method includes the steps of determining whether a first cryoprobe is connected to a rigid or pressure-resistant return run or a second cryoprobe is connected to a flexible or unpressurized return run; setting a first mode (counterpressure mode or back pressure regulating mode) if the first cryoprobe is connected and setting a second mode (free-running mode or front pressure regulating mode) if the second cryoprobe is connected; and controlling a pressure-setting device with a pressure regulating valve for regulating a pressure ratio between the admission run and return run of the cryoprobes. The step of setting the first or second modes includes a setting of a switching device in such a way that, in the first mode, at least the return run of the first cryoprobe is connected to a pressure regulating valve connection of the proportional valve and, in the second mode, at least the admission run of the second cryoprobe is connected to a pressure regulating valve connection of the proportional valve.

In this case too, therefore, the pressure ratio in the admission run and return run of the first cryoprobe or the second cryoprobe is advantageously regulated by means of a proportional valve.

### BRIEF DESCRIPTION OF THE DRAWINGS

The disclosed embodiments will be described in greater detail, pointing out further features and advantages, by reference to the example embodiments illustrated in the drawings.

FIG. 1 shows the components of a cryosurgical apparatus according to the disclosed embodiments.

FIG. 2 is a fluid circuit diagram for the cryosurgical apparatus according to a first disclosed embodiment.

FIG. 3 is a fluid flow diagram of the fluid circuit program from FIG. 2 at maximum cooling power for a flexible cryoprobe.

FIG. 4 is a fluid flow diagram of the fluid circuit diagram from FIG. 2 at maximum cooling power for a rigid cryoprobe.

FIG. 5 is a fluid circuit diagram for the cryosurgical apparatus according to a second disclosed embodiment.

FIG. 6 is a fluid flow diagram of the fluid circuit diagram from FIG. 5 at maximum cooling power for a flexible cryoprobe.

FIG. 7 is a fluid flow diagram of the fluid circuit diagram from FIG. 5 at maximum cooling power for a rigid cryoprobe.

FIG. 8 is a fluid circuit diagram for the cryosurgical apparatus according to a third disclosed embodiment

FIG. 9 is a fluid flow diagram of the fluid circuit diagram from FIG. 8 at maximum cooling power for a flexible cryoprobe.

FIG. 10 is a fluid flow diagram of the fluid circuit diagram from FIG. 8 at maximum cooling power for a rigid cryoprobe.

FIG. 11 is a fluid circuit diagram for the cryosurgical apparatus according to a fourth disclosed embodiment.

FIG. 12 is a fluid flow diagram of the fluid circuit diagram from FIG. 11 at maximum cooling power for a flexible cryoprobe.

FIG. 13 is a fluid flow diagram of the fluid circuit diagram from FIG. 11 at maximum cooling power for a rigid cryoprobe.

### DETAILED DESCRIPTION

The same reference numerals will be used in the following description for identical and equivalent parts.

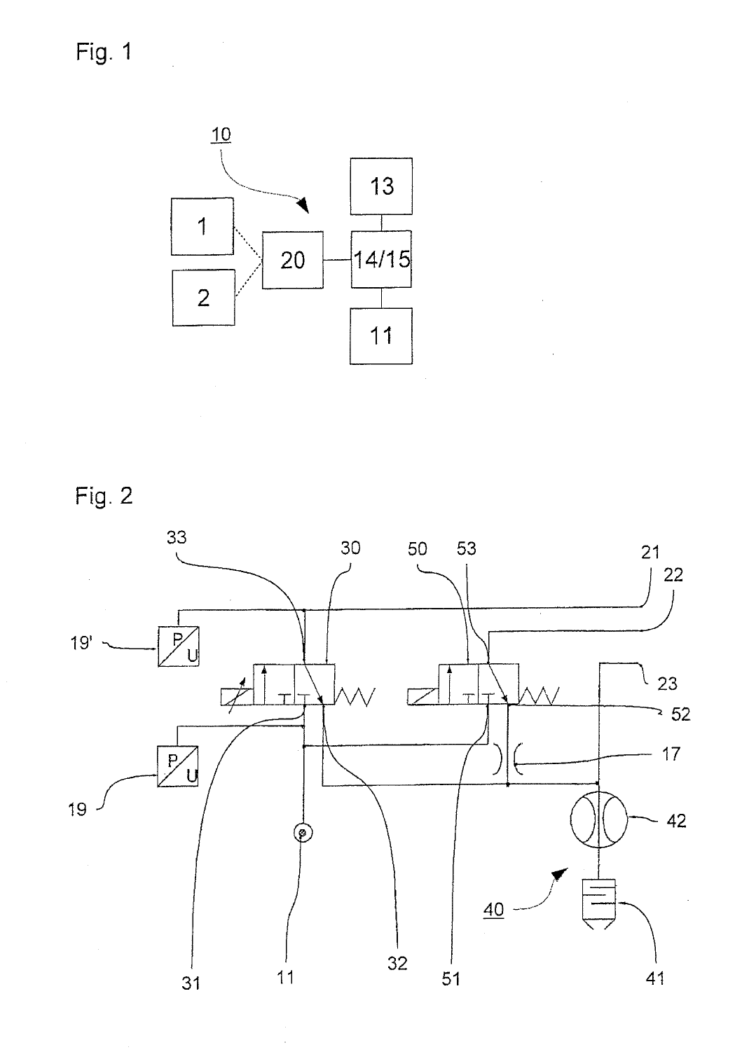

As seen in FIG. 1, a cryosurgical apparatus 10 according to the disclosed embodiments includes a fluid source 11 (for example a gas cylinder) for providing fluid at constant pressure, a switching device 15 and a pressure-setting device or means 14. The cryosurgical apparatus 10 also includes a controller 13 which controls the pressure-setting device 14 and the switching device 15 to operate connected cryoprobes 1, 2 with the necessary fluid. Both rigid cryoprobes 1 and flexible cryoprobes 2 can be connected to the cryosurgical apparatus 10 according to the disclosed embodiments, and the controller 13 regulates the pressure-setting device 14 and the switching device 15 accordingly.

### First Example Embodiment

FIG. 2 shows a configuration of the pressure-setting device 14 and the switching device 15, according to a first embodiment. The switching device includes a 3/2 proportional valve 30 and a 3/2 switching valve 50. A first proportional valve connection 31 of the proportional valve 30 is connected to the fluid source 11 and a second proportional valve connection 32 is connected to the ventilation/fluid removal means 40. The ventilation/fluid removal means 40 includes a sound absorber 41 and a flow sensor 42. The ventilation/fluid removal means 40 serves to drain the fluid. The third proportional valve connection 33 of the proportional valve 30 is connected to the first cryoprobe connection 21. The first cryoprobe connection 21 has a double function. In a first mode the return run of a rigid cryoprobe 1 is connected to the first cryoprobe connection 21 and in a second mode the admission run of a flexible cryoprobe 2 is connected to the first cryoprobe connection 21.

Furthermore, a first switching valve connection 51 of the 3/2 switching valve 50 is fluidly connected to the first proportional valve connection 31. The second switching valve connection 52 leads via a restrictor 17 to the ventilation means 40 and the second proportional valve connection 32. A third cryoprobe connection 23 is also linked to this line for the return run of the flexible cryoprobe 2. The third switching valve connection 53 is connected to a second cryoprobe connection 22 for the admission run of the rigid cryoprobe 1.

The controller 13 controls the compressed air setting device 14 and the switching device 15 according to FIG. 2 so that the rigid cryoprobe 1 can be connected in a first mode and a flexible cryoprobe 2 can be connected in a second mode. In order to be able to set the cooling power of the cryoprobes 1, 2 in accordance with a user input, the cryosurgical apparatus 10 according to the disclosed embodiments has sensors 19, 19′, 42 which determine the pressure at the first proportional valve connection 31 and at the third proportional valve connection 33. The sensors 19, 19′, 42 issue corresponding sensor signals to the controller 13 which sets the proportional valve 30 and the switching valve 50 using corresponding regulating signals. The proportional valve 30 and the switching valve 50 have appropriate actuators for this purpose.

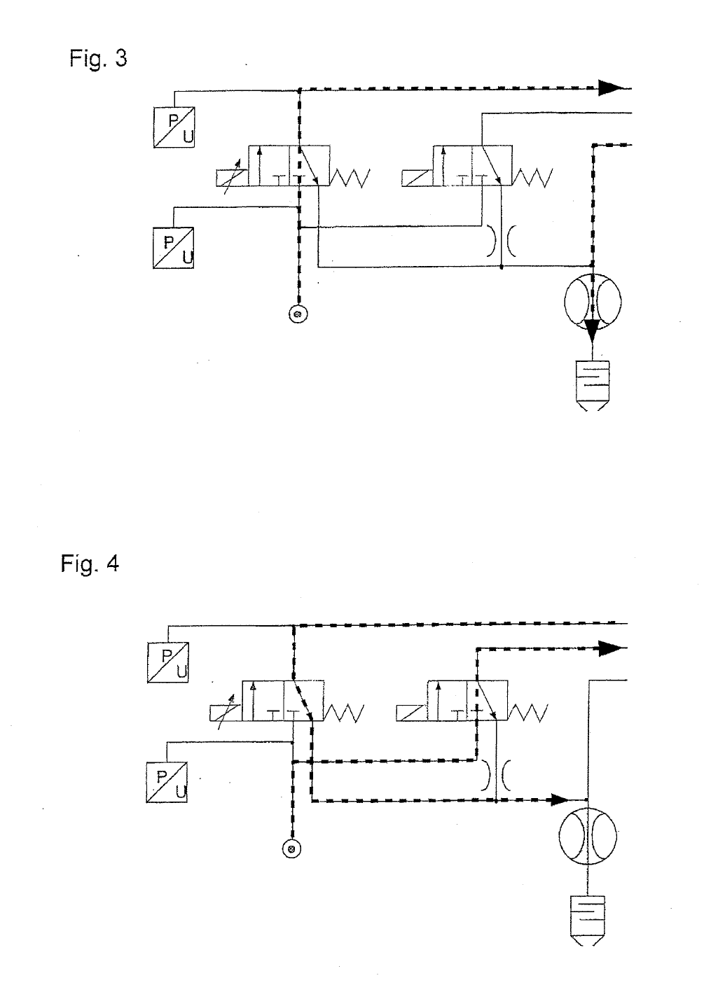

FIG. 3 shows the fluid flows in the second mode or in the front pressure regulating mode when the flexible cryoprobe 2 is connected and at maximum cooling power. The flexible cryoprobe 2 is connected to the first cryoprobe connection 21 and the third cryoprobe connection 23. The proportional valve 30 is activated by the controller 13 in such a way that the fluid from the fluid source 11 flows unrestricted via the first proportional valve connection 31 to the proportional valve 32 and from there into the admission run of the cryoprobe 2. The return run of the flexible cryoprobe 2 opens directly into the ventilation means 40. The switching valve 50 is deactivated (i.e. there is no fluid connection between the first switching valve connection 51 and the third switching valve connection 53). In order to avoid undesired rapid outflow of the fluid through the second cryoprobe connection 22, the restrictor 17 is provided there. The cooling power of the flexible cryoprobe 2 can be restricted by setting the proportional valve 30. As soon as the proportional valve 30 is deactivated (no fluid connection between the first and third proportional valve connection 30, 33), the flexible cryoprobe 2 is no longer cooled.

In the first mode or in the back pressure regulating mode (cf. FIG. 4), when the rigid cryoprobe 1 is connected and at maximum cooling power, the switching valve 50 is activated. There is thus a fluid connection between the second cryoprobe connection 22 and the fluid source 11. The fluid can flow unrestricted into the admission run of the rigid cryoprobe 1. The return run of the rigid cryoprobe 1 may be regulated by setting the proportional valve 30. As shown in FIG. 4, the maximum cooling power is achieved by deactivation of the proportional valve 30. In this valve position, the fluid flows unrestricted from the return run of the rigid cryoprobe 1 into the ventilation/fluid removal means 40. The outflow is measured by means of the flow sensor 42. Both the cooling power and the fluid flow may be restricted as a result of the setting of the proportional valve 30. In one position of the proportional valve 30 (intensive restriction), although the fluid continues to circulate, only an insignificant difference in pressure remains in the expansion chamber, so that cooling power is no longer produced here. If the temperature of the cryoprobe 1 is lower than that of the fluid, the fluid absorbs and discharges cold. The circulation of fluid therefore leads to defrosting of the rigid cryoprobe 1.

### Second Example Embodiment

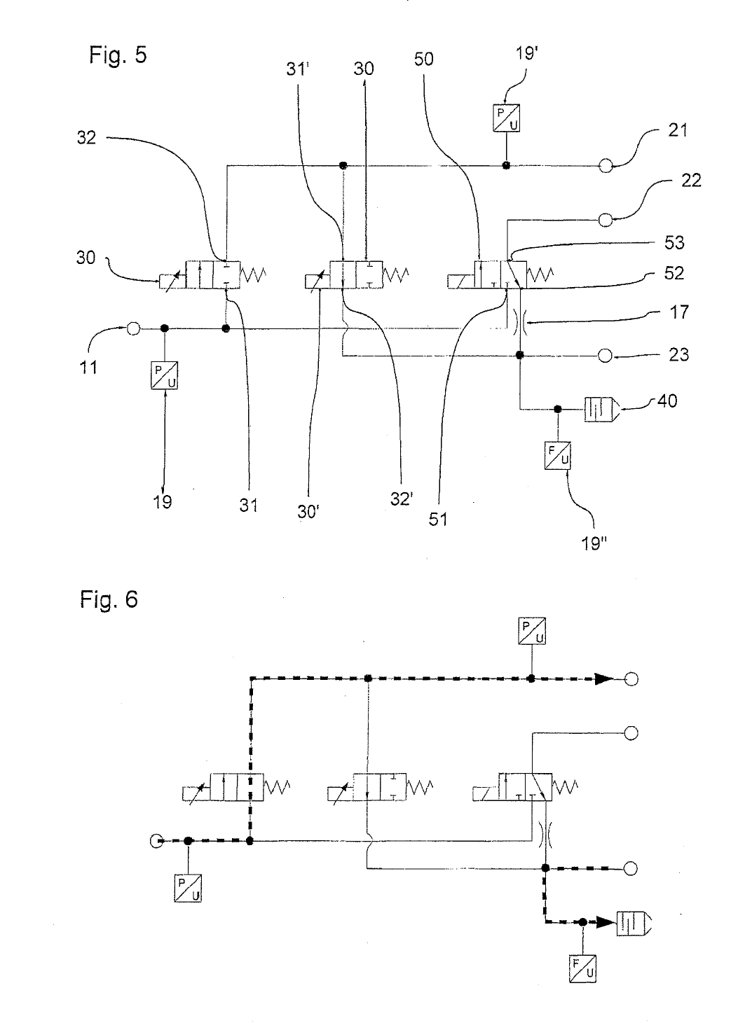

FIG. 5 shows a second fluid circuit which has a similar function to the described first fluid circuit. The 3/2 proportional valve 30 and the 3/2 switching valve 50 are however in this case replaced by two 2/2 proportional valves 30, 30′ and a 2/2 switching valve respectively. The first 2/2 proportional valve 30 has a first proportional valve connection 31 and a second proportional valve connection 32. The first proportional valve connection 31 is connected to the fluid source 11 and the second proportional valve connection 32 is connected to the first cryoprobe connection 21. The second proportional valve 30′ is also suspended by its first proportional valve connection 31′ from the first cryoprobe connection 21, while the second proportional valve connection 32′ is fluidly connected to the ventilation/fluid removal means 40. The third cryoprobe connection 23 and the second switching valve connection 52 are also connected to the ventilation/fluid removal means 40. The first switching valve connection 51 is connected to the fluid source 11 and the third switching valve connection 53 is connected to the second cryoprobe connection 22. Sensors 19, 19′, 19″ determined measured values at the fluid source 11 and the first cryoprobe connection 21 and the ventilation/fluid removal means 40 respectively.

In the second mode (cf. FIG. 6), for maximum cooling power of the flexible or second cryoprobe 2, the first proportional valve 30 is activated (open), the second proportional valve 30′ deactivated (closed) and the first switching valve 50 deactivated (fluid connection between the second and third switching valve connection 52, 53). The fluid flows from the fluid source 11 via the first proportional valve 30 to the first cryoprobe connection 21. The return run of the flexible cryoprobe 2 opens into the third cryoprobe connection 23 which is directly connected to the ventilation means 40. The power of the flexible cryoprobe 2 can be regulated by gradually deactivating the first proportional valve 30 which restricts the inflow of fluid depending on the position.

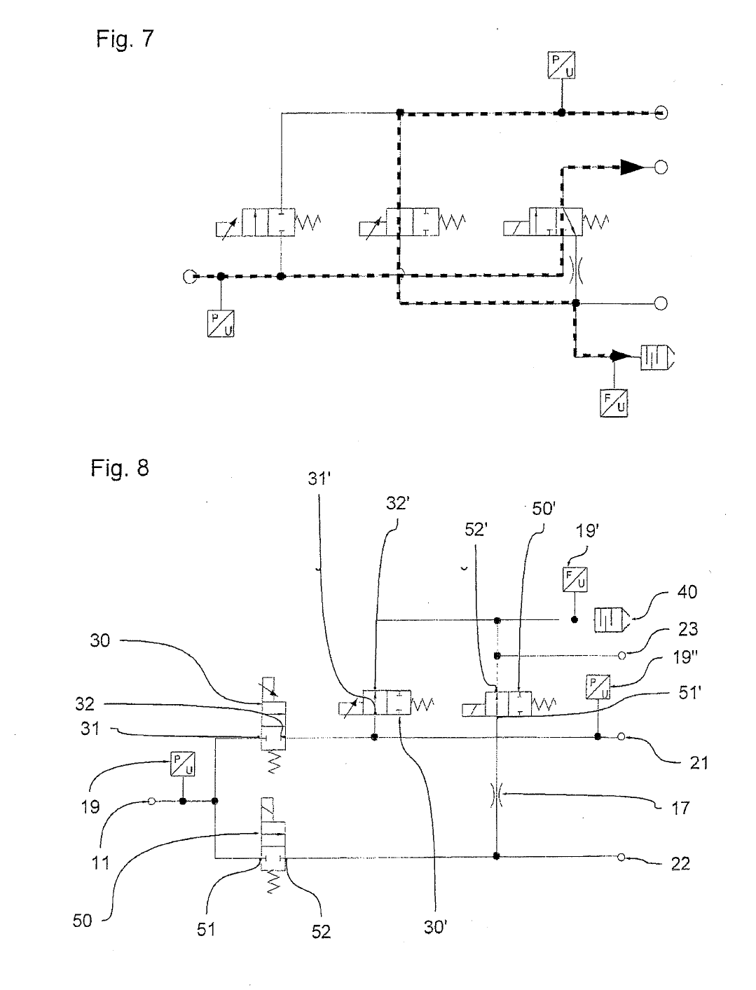

The maximum power in the first mode is, as shown in FIG. 7, achieved in that the first proportional valve 30 is deactivated, the second proportional valve 30′ activated and the switching valve 50 activated (fluid connection between the first and third switching valve connection 51, 53). The fluid therefore flows from the fluid source 11 unrestricted via the switching valve 50 into the second cryoprobe connection 22 and is removed from the first cryoprobe connection 21 via the second proportional valve 30′ to the ventilation means 40. The pressure at the first cryoprobe connection 21, and thus the cooling power of the first or rigid cryoprobe 1, may be set by way of the proportional valve 30′. The fluid circuit according to FIG. 5 also allows the flow of fluid in the first mode to be reversed in such a way that fluid is introduced into the rigid cryoprobe 1 via the return run or cryoprobe connection 21 and drained via the admission run or cryoprobe connection 22. The rigid cryoprobe 1 can thus be defrosted.

### Third Example Embodiment

FIG. 8 shows a functionally equivalent fluid circuit with two 2/2 switching valves 50, 50′ and two 2/2 proportional valves 30, 30′. The first proportional valve 30 and the first switching valve 50 are arranged parallel to each other, the first proportional valve connection 31 and the first switching valve connection 51 being fluidly connected to the fluid source 11. The second proportional valve connection 32 of the first proportional valve 30 is connected to the first proportional valve connection 31′ of the second proportional valve 30′ and the first cryoprobe connection 21. The second switching valve connection 52 of the first switching valve 50 is connected to the second cryoprobe connection 22 and, via a restrictor 17, to the first switching valve connection 51′ of the second switching valve 50′. The second proportional valve connection 32′ of the second proportional valve 30′, the second switching valve connection 52′ of the second switching valve 50′ and the third cryoprobe connection 23 are fluidly connected to the ventilation/fluid removal means 40. Sensors 19 to 19″ are accordingly provided.

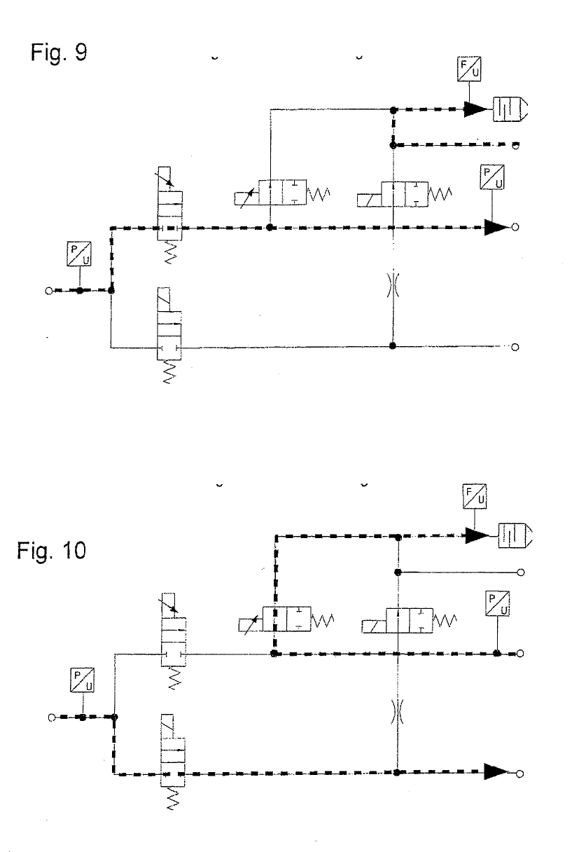

In the second mode the first proportional valve 30 is, as shown in FIG. 9, activated (fully opened) and the second proportional valve 30′ is deactivated (closed) at full power. The first and second switching valves 50, 50′ are deactivated in this mode. The fluid flows from the fluid source 11 via the first proportional valve 30 to the first cryoprobe connection 21 and is removed from the third cryoprobe connection 23 to the ventilation/fluid removal means 40. In the second mode the fluid pressure applied to the first cryoprobe connection 21, and thus the cooling power, may be set by regulating the first proportional valve 30.

In the first mode, at maximum power, the first proportional valve 30 is deactivated, the second proportional valve 30′ activated, the first switching valve 50 activated and the second switching valve 50′ deactivated (cf. FIG. 10). The fluid flows unrestricted from the fluid source 11 to the second cryoprobe connection 22 and from the first cryoprobe connection 21 via the second proportional valve 30′ to the ventilation means 40. The power of the first cryoprobe 1 may be set by restricting the outflowing flow of fluid by means of the second proportional valve 30′.

### Fourth Example Embodiment

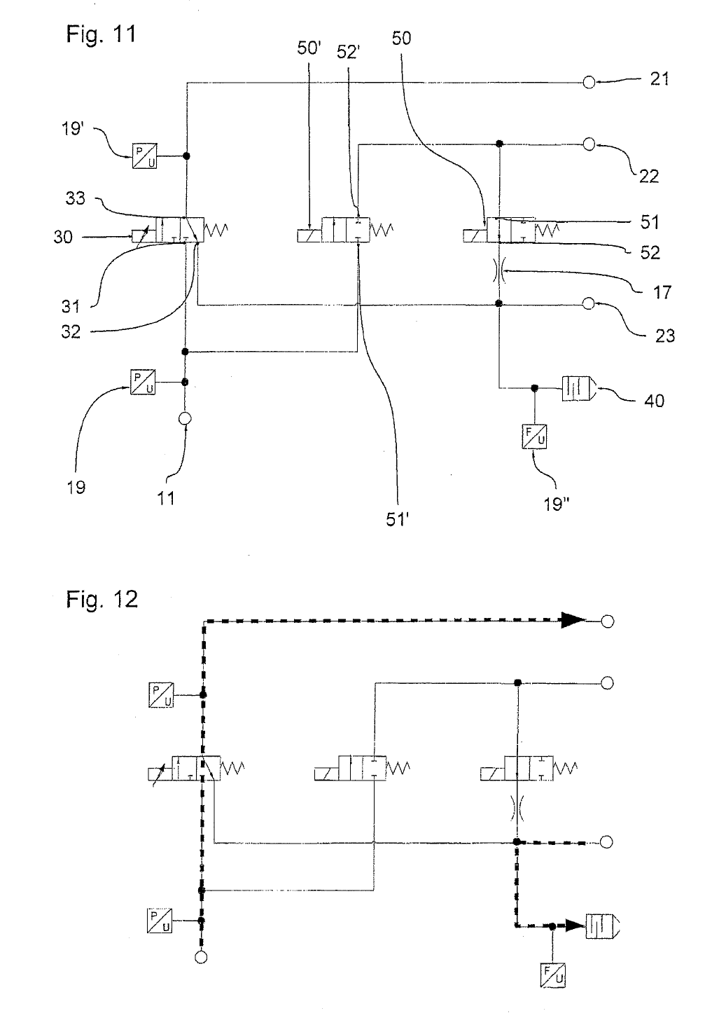

The fourth example embodiment according to FIG. 11 has a 3/2 proportional valve 30 and two 2/2 switching valves 50, 50′. The first proportional valve connection 31 is connected to the fluid source 11, the second proportional valve connection 32 to the ventilation/fluid removal means 40 and the third proportional valve connection 33 of the proportional valve 30 to the first cryoprobe connection 21. There are further connections between the second cryoprobe connection 22 and the first switching valve connection 51 of the first switching valve 50 and also the second switching valve connection 52′ of the second switching valve 50′. The second switching valve connection 52 of the first switching valve 50 is connected to the ventilation/fluid removal means 40 and the first switching valve connection 51 of the second switching valve 50′ is connected to the fluid source 11. There is a fluid connection between the third cryoprobe connection 23 and the ventilation/fluid removal means 40. Sensors 19, 19′, 19″ are provided at the fluid source 11, the first cryoprobe connection 21 and the ventilation/fluid removal means 40. A restrictor 17 is connected downstream of the second switching valve connection 52 of the first switching valve 50.

In the second mode, at maximum power, the first proportional valve 30 is activated (unrestricted connection between the first and third proportional valve connections 31, 33) and the two switching valves 50, 50′ are deactivated (cf. FIG. 12). The fluid flows unrestricted from the fluid source 11 via the proportional valve 30 to the first cryoprobe connection 21 and from the second cryoprobe connection 22 into the ventilation/fluid removal means 40. The flow of fluid can be regulated via a restriction in the proportional valve 30.

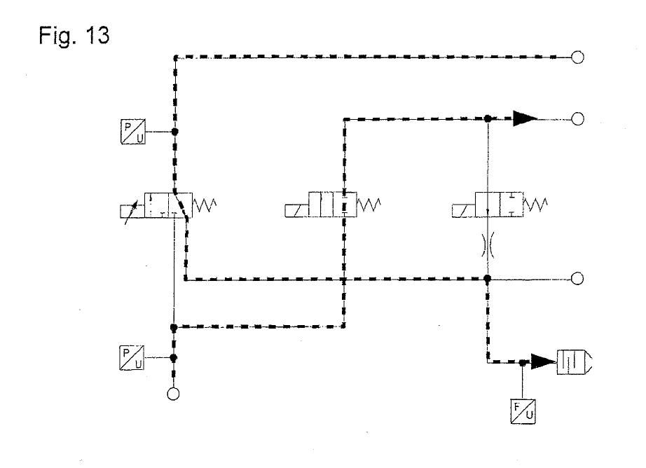

In the first mode (cf. FIG. 13), at maximum power, the proportional valve 30 is deactivated (unrestricted connection between the first and third proportional valve connections 32, 33), the first switching valve 50 is deactivated and the second switching valve 50′ is activated. The fluid flows from the fluid source 11 unrestricted into the second cryoprobe connection 22 (via the second switching valve 50′) and from the first cryoprobe connection 21 via the proportional valve 30 into the ventilation/fluid removal means 40. The return run from the first cryoprobe 1 can be regulated by setting the proportional valve 30. The proportional valve 30 then serves as a restrictor and increases the pressure in the return run line. The power of the rigid cryoprobe 1 thus decreases.

The example embodiments described hereinbefore serve merely to illustrate different circuit arrangements which achieve the claimed effect. For marketable implementation, it may be necessary to provide further restrictors 17 or check valves. Furthermore, it is possible to combine the second and third cryoprobe connections 22, 23 by means of a further 3/2 switching valve into one connection.

The person skilled in the art will be familiar with numerous further embodiments of the circuits that achieve the same effect.

1-16. (canceled)

17. A cryosurgical apparatus for operating a first cryoprobe with a pressure-resistant return run and a second cryoprobe with an unpressurized return run, wherein the apparatus has a first mode for operating the first cryoprobe and a second mode for operating the second cryoprobe, the apparatus comprising:
a controller;
a fluid source for providing a gas, which can be introduced into the cryoprobes for cooling the cryoprobes by means of the Joule-Thomson effect and/or by evaporating a liquefied gas;
at least two cryoprobe connections, wherein, in the first mode, a return run of the first cryoprobe is connected to one of the cryoprobe connections and, in the second mode, an admission run of the second cryoprobe is connected to the same one of the cryoprobe connections; and
a pressure-setting device including at least one pressure regulating valve, wherein the pressure regulating valve is connected to at least one of the cryoprobe connections in such a way that the pressure regulating valve can be used to regulate, in the first and second modes, the pressure ratio of an admission run to the return run of the first cryoprobe and the pressure in the admission run of the second cryoprobe, respectively, and
wherein the controller controls a cooling power of the first and second cryoprobes by setting of the pressure regulating valve.

18. The cryosurgical apparatus according to claim 17, wherein the first mode is a back pressure regulating mode and the second mode is a front pressure regulating mode.

19. The cryosurgical apparatus according to claim 17, wherein, in the first mode, the pressure-setting device is connected to the first cryoprobe to allow the pressure in the admission run and in the return run of the first cryoprobe to be regulated by setting the pressure regulating valve.

20. The cryosurgical apparatus according to claim 17, wherein, in the second mode, the pressure-setting device is connected to the second cryoprobe to allow, in the case of a substantially open or unpressurized return run of the second cryoprobe, the pressure in the admission run of the second cryoprobe to be regulated.

21. The cryosurgical apparatus according to claim 17, wherein the pressure-setting device comprises a 3/2 proportional valve connected by a first proportional valve connection to the fluid source, by a second proportional valve connection to a fluid removal means and by a third proportional valve connection to at least one of the cryoprobe connections.

22. The cryosurgical apparatus according to claim 17, further comprising a switching device with at least one switching valve which is connected to the pressure-setting means and at least one cryoprobe connection for changing between the first and the second mode.

23. The cryosurgical apparatus according to claim 22, wherein the controller comprises actuators and sets the first or the second mode.

24. The cryosurgical apparatus according to claim 22, wherein the switching device comprises a 3/2 switching valve connected by a first switching valve connection to the pressure-setting device, by a second switching valve connection to a ventilation means and by the third switching valve connection to at least one of the cryoprobe connections.

25. The cryosurgical apparatus according to claim 17, further comprising at least one pressure sensor for determining an input pressure and/or an output pressure of the pressure-setting device, the at least one pressure sensor providing pressure sensor signals which are used by the controller for regulating the pressure ratio between the admission run and the return run or for regulating the pressure in the admission run.

26. The cryosurgical apparatus according to claim 17, wherein the fluid is a gas mixture of carbon dioxide (CO₂) and/or nitrous oxide (N₂O).

27. The cryosurgical apparatus according to claim 17, further comprising a detection means which ascertains whether the first cryoprobe or the second cryoprobe is connected to the cryoprobe connections.

28. The cryosurgical apparatus according to claim 27, wherein the detection means reads out an apparatus marker of the cryoprobes and the controller is connected to the detection means in order to receive apparatus mark signals from the detection means and to set the first or the second mode accordingly.

29. The cryosurgical apparatus according to claim 17, further comprising a flow sensor for determining the cooling power of the first or second cryoprobe.

30. The cryosurgical apparatus according to claim 17, further comprising a securing unit for detecting a pneumatic connection between at least one cryoprobe connection and the first or second cryoprobe, the securing means interrupting a supply of fluid to the at least one cryoprobe connection if the pneumatic connection is interrupted.

31. A method for operating a cryoprobe with a cryosurgical apparatus, wherein the apparatus provides a fluid for cooling the cryoprobe, the method comprising the steps of:
determining whether a first cryoprobe is connected to a pressure-resistant return run or a second cryoprobe is connected to an unpressurized return run;
setting a first mode if the first cryoprobe is connected and setting a second mode if the second cryoprobe is connected; and
controlling a pressure-setting device with a pressure regulating valve for regulating, in the first mode, a pressure ratio between an admission run and the return run of the first cryoprobe and regulating, in the second mode, the pressure in the admission run of the second cryoprobe,
wherein the step of setting the first or second mode includes setting a switching device such that, in the first mode, at least the return run of the first cryoprobe is connected to a pressure regulating valve connection of the pressure regulating valve and, in the second mode, at least the admission run of the second cryoprobe is connected to a pressure regulating valve connection of the pressure regulating valve.

32. The method according to claim 31, wherein the first mode is a back pressure regulating mode and the second mode is a front pressure regulating mode.

33. The method according to claim 31, wherein in the first mode for setting the cooling power of the first cryoprobe, the pressure in the return run is regulated at constant pressure in the admission run.

34. The method according to claim 31, wherein in the second mode for setting the cooling power of the second cryoprobe, the pressure in the admission run of the second cryoprobe is regulated.
